(19) United States
(12) Patent Application Publication
(10) Pub. No.: US 2019/0351263 A1
(43) Pub. Date: Nov. 21, 2019

(54) DISPOSABLE WIPE FOR CLEANING DENTURES

(71) Applicant: Ricky Allen Pace, Ocean Springs, MS (US)

(72) Inventor: Ricky Allen Pace, Ocean Springs, MS (US)

(21) Appl. No.: 16/415,802

(22) Filed: May 17, 2019

Related U.S. Application Data
(60) Provisional application No. 62/672,936, filed on May 17, 2018.

(51) Int. Cl. A61Q 11/02; A61C 17/00; A61K 8/02

(52) U.S. Cl. CPC A61Q 11/02; A61K 8/0208; A61C 17/036

(57) ABSTRACT

A denture cleaning wipe is a sheet made of a flexible material with a gripping surface opposite a cleaning surface. A first finger pocket and a second finger pocket are each coupled to the gripping surface so that a first portion of an outer edge of the first and second finger pockets are coupled to the sheet and a second portion of the outer edge of the first and second finger pockets are detached from the sheet defining entrances into the first and second finger pockets. The first finger pocket and the second finger pocket are spaced apart and oriented so that the entrances are facing each other.

### CROSS-REFERENCE TO RELATED APPLICATION

This application claims the benefit of priority of U.S. provisional application No. 62/672,936, filed May 17, 2018, the contents of which are herein incorporated by reference.

### BACKGROUND OF THE INVENTION

The present invention relates to cleaning dentures and, more particularly, to an improved wipe to remove denture adhesive from dentures.

Dentures (also known as false teeth) are prosthetic devices constructed to replace missing teeth, and are supported by the surrounding soft and hard tissues of the oral cavity. Denture adhesives are used to adhere the dentures to the user's gums. Currently, to remove denture adhesives from the dentures, a user may use a wash cloth, toilet paper, or paper towels, which are ineffective.

As can be seen, there is a need for an improved wipe to remove denture adhesive from dentures.

### SUMMARY OF THE INVENTION

In one aspect of the present invention, a denture cleaning wipe comprises: a sheet made of a flexible material and comprising a gripping surface opposite a cleaning surface; a first finger pocket coupled to the gripping surface so that a first portion of an outer edge of the first finger pocket is coupled to the sheet and a second portion of the outer edge of the first finger pocket is detached from the sheet defining a first entrance into the first finger pocket; and a second finger pocket coupled to the gripping surface so that a first portion of an outer edge of the second finger pocket is coupled to the sheet and a second portion of the outer edge of the second finger pocket is detached from the sheet defining a second entrance into the second finger pocket, wherein the first finger pocket and the second finger pocket are spaced apart and oriented so that the first entrance is facing the second entrance.

In another aspect of the present invention, a method of cleaning dentures comprises: providing denture cleaning wipe comprising: a sheet made of a flexible material and comprising a gripping surface opposite a cleaning surface; a first finger pocket coupled to the gripping surface so that a first portion of an outer edge of the first finger pocket is coupled to the sheet and a second portion of the outer edge of the first finger pocket is detached from the sheet defining a first entrance into the first finger pocket; and a second finger pocket coupled to the gripping surface so that a first portion of an outer edge of the second finger pocket is coupled to the sheet and a second portion of the outer edge of the second finger pocket is detached from the sheet defining a second entrance into the second finger pocket, wherein the first finger pocket and the second finger pocket are spaced apart and oriented so that the first entrance is facing the second entrance; placing a finger into one of the first finger pocket and the second finger pocket; and wiping dentures with the cleaning surface of the denture cleaning wipe.

These and other features, aspects and advantages of the present invention will become better understood with reference to the following drawings, description and claims.

### BRIEF DESCRIPTION OF THE DRAWINGS

FIG. 1 is a perspective view of an embodiment of the present invention;

FIG. 2 is an exploded view of an embodiment of the present invention;

FIG. 3 is a cross-sectional view taken along line 3-3 of FIG. 1;

FIG. 4 is a perspective view of an embodiment of the present invention, shown in use;

FIG. 5 is a perspective view of an embodiment of the present invention, shown in use;

FIG. 6 is a cross-sectional view taken along line 6-6 of FIG. 4; and

FIG. 7 is a perspective view of an embodiment of the present invention shown in use.

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

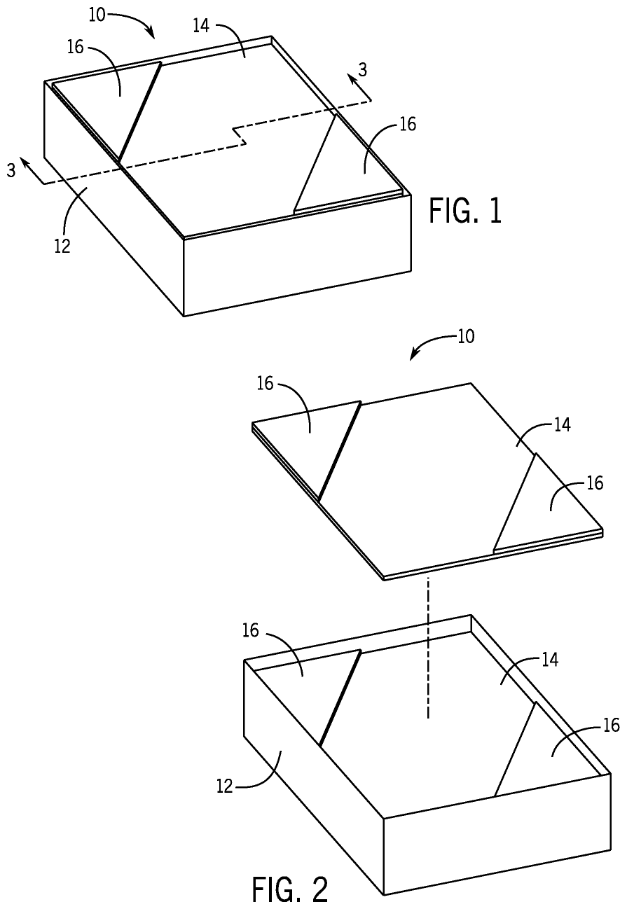

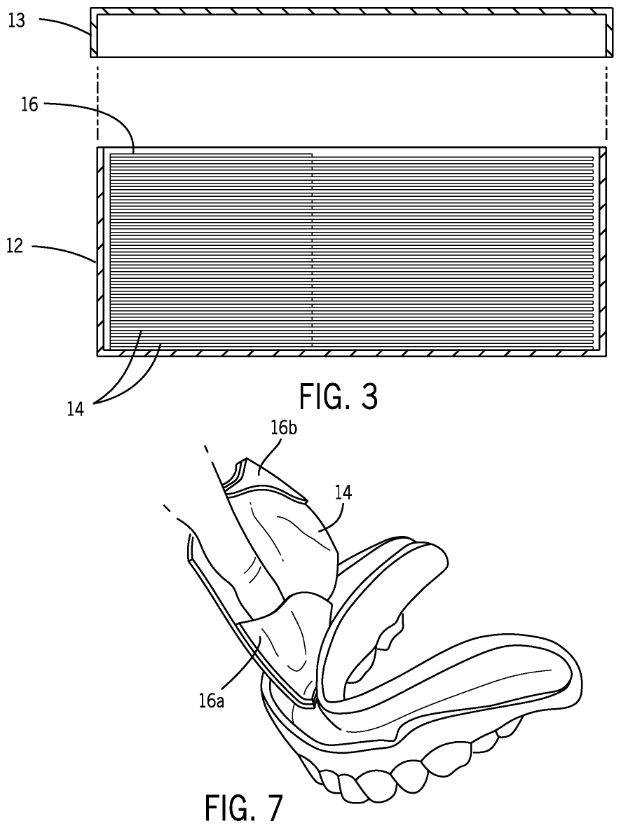

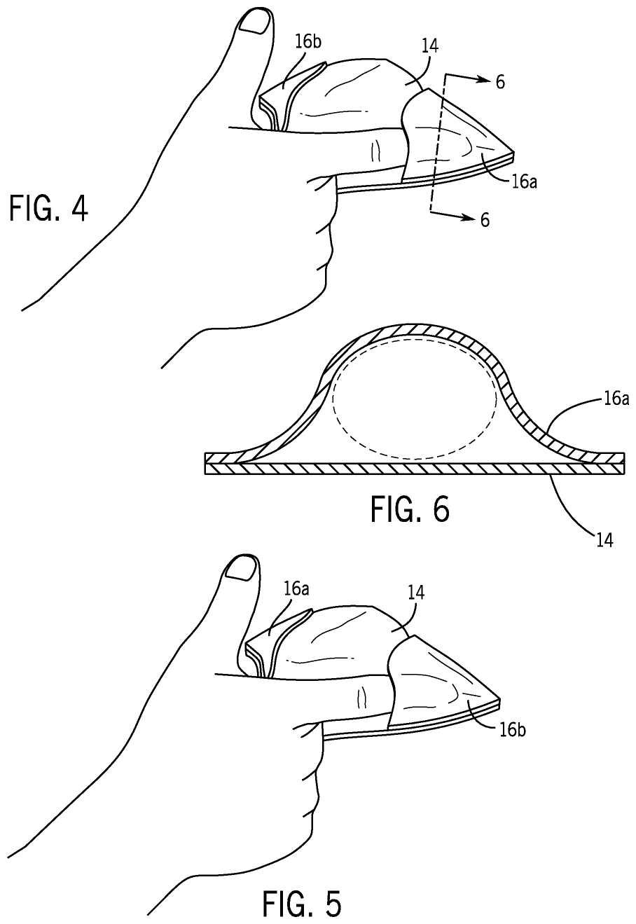

Referring to FIGS. 1 through 7, the present invention includes a denture cleaning wipe 10. The denture cleaning wipe 10 is a sheet 14 made of a flexible material with a gripping surface opposite a cleaning surface. The sheet 14 includes finger pockets 16. A first finger pocket 16a is coupled to the gripping surface so that a first portion of an outer edge of the first finger pocket 16a is coupled to the sheet 14 and a second portion of the outer edge of the first finger pocket 16a is detached from the sheet defining a first entrance into the first finger pocket 16a. A second finger pocket 16b is coupled to the gripping surface so that a first portion of an outer edge of the second finger pocket 16b is coupled to the sheet 14 and a second portion of the outer edge of the second finger pocket 16b is detached from the sheet 14 defining a second entrance into the second finger pocket 16b. The first finger pocket 16a and the second finger pocket 16b are spaced apart and oriented so that the first entrance is facing the second entrance.

The sheet 14 of the present invention may have a rectangular or square shape with four corners. In such embodiments, the first finger pocket 16a is coupled to a first corner of the sheet 14 and the second finger pocket 16b is coupled to a second corner of the sheet 14 opposite the first corner. In certain embodiments, the first finger pocket 16a and the second finger pocket 16b each comprise a triangular shape. In such embodiments, the entrances may be wide enough to easily fit different sized fingers therein. Additionally, the fingertips of a user are disposed at the corners of the sheet 14 and finger pockets 16, allowing a user to easily maneuver the sheet 14 in order to clean the dentures.

A method of cleaning dentures may include the following. Provide a denture cleaning wipe 10 as described above. Place a first finger into the first finger pocket 16a and a second finger in the second finger pocket 16b. For example, the user may place their thumb in the first finger pocket 16a and their pointer finger in the second finger pocket 16b. Wipe dentures with the cleaning surface of the denture cleaning wipe 10. A plurality of denture cleaning wipes 10 may be stored in a box 12 with a lid 13.

It should be understood, of course, that the foregoing relates to exemplary embodiments of the invention and that modifications may be made without departing from the spirit and scope of the invention as set forth in the following claims.

What is claimed is:

1. A denture cleaning wipe comprising:
a sheet made of a flexible material and comprising a gripping surface opposite a cleaning surface;
a first finger pocket coupled to the gripping surface so that a first portion of an outer edge of the first finger pocket is coupled to the sheet and a second portion of the outer edge of the first finger pocket is detached from the sheet defining a first entrance into the first finger pocket; and
a second finger pocket coupled to the gripping surface so that a first portion of an outer edge of the second finger pocket is coupled to the sheet and a second portion of the outer edge of the second finger pocket is detached from the sheet defining a second entrance into the second finger pocket, wherein
the first finger pocket and the second finger pocket are spaced apart and oriented so that the first entrance is facing the second entrance.

2. The denture cleaning wipe of claim 1, wherein the first finger pocket is coupled to a first corner of the sheet and the second finger pocket is coupled to a second corner of the sheet opposite the first corner.

3. The denture cleaning wipe of claim 1, wherein the sheet comprises a square shape.

4. The denture cleaning wipe of claim 3, wherein the first finger pocket and the second finger pocket each comprise a triangular shape.

5. A method of cleaning dentures comprising:
providing denture cleaning wipe comprising:
a sheet made of a flexible material and comprising a gripping surface opposite a cleaning surface;
a first finger pocket coupled to the gripping surface so that a first portion of an outer edge of the first finger pocket is coupled to the sheet and a second portion of the outer edge of the first finger pocket is detached from the sheet defining a first entrance into the first finger pocket; and
a second finger pocket coupled to the gripping surface so that a first portion of an outer edge of the second finger pocket is coupled to the sheet and a second portion of the outer edge of the second finger pocket is detached from the sheet defining a second entrance into the second finger pocket, wherein
the first finger pocket and the second finger pocket are spaced apart and oriented so that the first entrance is facing the second entrance;
placing a finger into one of the first finger pocket and the second finger pocket; and
wiping dentures with the cleaning surface of the denture cleaning wipe.

6. The method of claim 5, wherein a plurality of denture cleaning wipes are stored in a box.

7. The method of claim 5, wherein the first finger pocket is coupled to a first corner of the sheet and the second finger pocket is coupled to a second corner of the sheet opposite the first corner.

8. The method of claim 7, wherein the sheet comprises a square shape.

9. The method of claim 8, wherein the first finger pocket and the second finger pocket each comprise a triangular shape.
